(19) United States
(12) Patent Application Publication
(10) Pub. No.: US 2024/0415789 A1
(43) Pub. Date: Dec. 19, 2024

(54) CYCLOBENZAPRINE FOR TREATMENT OR PREVENTION OF SEXUAL DYSFUNCTION ASSOCIATED WITH MENTAL HEALTH CONDITIONS IN FEMALE PATIENTS

(71) Applicants: Tonix Pharmaceuticals Holding Corp., Chatham, NJ (US); Tonix Pharmaceuticals, Inc., Chatham, NJ (US)

(72) Inventors: Megan Elizabeth Parmenter, Mineola, NY (US); Gregory M. Sullivan, Barryville, NY (US)

(21) Appl. No.: 18/698,483

(22) PCT Filed: Oct. 5, 2022
(86) PCT No.: PCT/US2022/045791

§ 371 Date: Apr. 4, 2024

Related U.S. Application Data
(60) Provisional application No. 63/252,964, filed on Oct. 6, 2021.

(51) Int. Cl. A61K 31/135; A61K 9/00; A61K 45/06; A61K 47/26; A61P 15/00

(52) U.S. Cl. CPC A61K 31/135; A61K 9/0056; A61K 45/06; A61K 47/26; A61P 15/00

(57) ABSTRACT

The present invention concerns methods of treating and/or preventing sexual dysfunction, wherein the sexual dysfunction is associated with one or more mental health conditions, the method using a pharmaceutical composition comprising therapeutically effective amounts of cyclobenzaprine or a pharmaceutically acceptable salt thereof, and potentially one or more additional agents.

### CROSS-REFERENCE TO RELATED APPLICATIONS

This application claims priority to and benefit from U.S. Provisional Application No. 63/252,964, filed Oct. 6, 2021, the entire disclosure of which is hereby incorporated by reference.

### BACKGROUND

There is increasing documentation of the comorbidity of sexual dysfunction (SD) and posttraumatic stress disorder (PTSD) among veterans (Letica-Crepulja et al., 2019). Compared to those without any mental health diagnosis or with a mental health diagnosis other than PTSD, veterans with PTSD are at a higher risk of SD. This is true regardless of chronicity of symptoms, age or other health concerns, suggesting that the mechanisms underlying such dysfunction are directly related to PTSD (Hirsch, 2009). Common types of SD in males include erectile dysfunction, sexual disinterest, and premature ejaculation;

whereas common types of SD in females include fear of sex, arousal problems, orgasm problems, sexual disinterest, and vaginal pain. Moreover, the relationship between sexual functioning and psychological well-being is stronger for females than males (Rosen & Bauchman, 2008).

Despite the importance of addressing co-morbid symptoms, the topic of SD is often overlooked clinically and underexamined in the research literature (Tran et al., 2015). In terms of treatment, experts recommend that evidence-based treatment for PTSD should be the first priority. However, treating PTSD does not necessarily resolve symptoms of SD. For example, when compared with a no-treatment control group, women who participated in cognitive behavioral therapy for PTSD showed significant reductions in symptoms of PTSD but did not show significant improvements in sexual functioning (Cohen & Hien, 2006). In addition, serotonin reuptake inhibitors (SSRIs) and benzodiazepines, commonly prescribed psychiatric medications for the treatment of PTSD, are shown to exacerbate pre-existing SD, as well as contribute to poor compliance and refusal of medication (Keller, McGarvey, Clayton, 2006).

Cyclobenzaprine and its pharmaceutically acceptable salts is a serotonin-2A, alpha-1-adrenergic, and histamine-1 receptor antagonist that is being developed to target sleep disturbance and hyperarousal in PTSD as a treatment for PTSD. A Phase 2 study of cyclobenzaprine HCl in military-related PTSD had very low rates of adverse events related to sexual function in both the drug and placebo groups. Therefore, in the subsequent Phase 3 study in military-related PTSD, systematic study was undertaken to assess the effects of treatment on female and male sexual functioning.

### SUMMARY OF THE DISCLOSURE

Some embodiments of this disclosure are:
1. A method for treating or preventing sexual dysfunction and associated symptoms thereof, comprising administering to a female subject in need or at risk thereof, a pharmaceutical composition comprising a therapeutically effective amount of cyclobenzaprine and a pharmaceutically acceptable carrier, wherein the sexual dysfunction is associated with one or more mental health conditions selected from the group consisting of a psychological condition, wherein the psychological condition is neglect, a mood disorder, wherein the mood disorder is a substance related and addictive disorder, a trauma and stressor related disorder, wherein the trauma and stressor related disorder is a disinhibited social engagement disorder, a personality disorder, a somatic symptom disorder, and an obsessive compulsive disorder.
2. The method of embodiment 1, wherein the cyclobenzaprine is a free base or a pharmaceutically acceptable salt thereof.
3. The method of embodiment 1 or 2, wherein the pharmaceutically acceptable salt of cyclobenzaprine is a cyclobenzaprine acid salt.
4. The method of embodiment 3, wherein the cyclobenzaprine acid salt is cyclobenzaprine HCl.
5. The method of any one of embodiments 1-4, wherein the cyclobenzaprine or pharmaceutically salt thereof is in the form of a eutectic.
6. The method of embodiment 5, wherein the eutectic is a mannitol eutectic.
7. The method of embodiment 6, wherein the mannitol eutectic is selected for the group consisting of a 75%+2% cyclobenzaprine HCl and 25%+2% mannitol eutectic, a 65%+2% cyclobenzaprine HCl and 35%+2% 8-mannitol eutectic, a mixture of a 75%+2% cyclobenzaprine HCl and 25%+2% β-mannitol and a 65%+2% cyclobenzaprine HCl and 35%+2% 8-mannitol eutectic, and a granule comprising an outer layer of a 65%+2% cyclobenzaprine HCl and 35%+2% 8-mannitol eutectic and an inner layer of β-mannitol.
8. The method of any one of embodiments 1-7, wherein the composition comprising a pharmaceutically acceptable salt of cyclobenzaprine further comprises a basifying agent.
9. The method of embodiment 8, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
10. The method according to embodiment 9, wherein the basifying agent is dipotassium hydrogen phosphate.
11. The method of embodiment 1, wherein the composition comprises between 0.1 mg and 30 mg of cyclobenzaprine or a pharmaceutically acceptable salt thereof.
12. The method of embodiment 11, wherein the composition comprises between 1 mg and 20 mg of cyclobenzaprine or a pharmaceutically acceptable salt thereof.
13. The method of embodiment 12, wherein the composition comprises less than 10 mg of cyclobenzaprine or a pharmaceutically acceptable salt thereof.
14. The method of embodiment 12, wherein the composition comprises less than 5 mg of cyclobenzaprine or a pharmaceutically acceptable salt thereof.
15. The method of embodiment 13, wherein the composition comprises about 5.6 mg of cyclobenzaprine or a pharmaceutically acceptable salt thereof.
16. The method of embodiment 15, wherein the composition comprises about 5.6 mg of cyclobenzaprine HCl.
17. The method of embodiment 13, wherein the composition comprises about 2.8 mg of cyclobenzaprine or a pharmaceutically acceptable salt thereof.
18. The method of embodiment 17, wherein the composition comprises about 2.8 mg of cyclobenzaprine HCl.
19. The method of embodiment 17 or 18, wherein the composition is administered simultaneously or sequentially in two dosage units, and wherein the combined amount of the composition in the two dosage units is about 5.6 mg of cyclobenzaprine or a pharmaceutically acceptable salt.
20. The method of embodiment 19, wherein the composition is administered simultaneously in two dosage units, and wherein each dosage unit comprises about 2.8 mg of cyclobenzaprine or a pharmaceutically acceptable salt thereof.
21. The method of embodiment 19, wherein the composition is administered sequentially in two dosage units, and wherein each dosage unit comprises about 2.8 mg of cyclobenzaprine or a pharmaceutically acceptable salt thereof.
22. The method of any one of embodiments 1-21, wherein the pharmaceutical composition is administered daily.
23. The method of any one of embodiments 1-22, wherein the composition is administered once daily.
24. The method of embodiment 23, wherein the pharmaceutical composition is formulated for sublingual, buccal, oral, intravenous, intramuscular, subcutaneous, inhalational, intranasal, transdermal, parenteral, rectal, or vaginal administration.
25. The method of embodiment 23, wherein the pharmaceutical composition is formulated as a suppository or a thin film dosage form.
26. The method of embodiment 25, wherein the pharmaceutical composition is formulated as a thin film dosage form.
27. The method of embodiment 24 or 26, wherein the pharmaceutical composition is formulated for sublingual administration.
28. The method of any one of embodiments 1-27, wherein the method further comprises administering sequentially or simultaneously one or more therapeutic agents selected from the group consisting an estrogen receptor modulator, a 5-hydroxytryptamine 1A (5-HT_{1A}) receptor agonist, a 5-hydroxytryptamine 2A (5-HT_{2A}) antagonist, a synthetic or gonadal steroid agent, a phosphodiesterase inhibitor, a melanocortin receptor agonist, an alpha-1-adrenergic receptor antagonist, a beta-adrenergic antagonist, an anticonvulsant or a mood stabilizer, a selective serotonin reuptake inhibitor, a serotonin-norepinephrine reuptake inhibitor, an antidepressant, an anti-anxiety agent, an antipsychotic, an antihistamine, a benzodiazepine, a psychoactive agent, a barbiturate, lithium, an antihypertensive agent, an antilipid agent, a hormonal agent, a gonadotropin-releasing hormone (GnRh) agonist, a contraceptive agent, an anticholinergic agent, an amphetamine, a dopaminergic receptor agonist, an anorexic agent, and a narcotic agent.
29. The method of embodiment 28, wherein the estrogen receptor modulator is ospemifene.
30. The method of embodiment 28, wherein the 5-HT_{1A} receptor agonist or the 5-HT_{2A} receptor agonist is flibanserin.
31. The method of embodiment 28, wherein the dopaminergic receptor agonist is apomorphine.
32. The method of embodiment 28, wherein the steroid agent is tibolone, estrogen, or testosterone.
33. The method of embodiment 28, wherein the phosphodiesterase inhibitor is sildenafil or tadalafil.
34. The method of embodiment 28, wherein the melanocortin receptor agonist is bremelanotide.
35. The method of embodiment 28, wherein the alpha-1-adrenergic receptor antagonist is prazosin, terazosin, doxazosin, silodosin, alfuzosin, or tamsulosin.
36. The method of embodiment 28, wherein the beta adrenergic receptor antagonist is propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, ICI-118,551, SR 59230A, or nebivolol.
37. The method of embodiment 28, wherein the anticonvulsant or mood stabilizer is carbamazepine, divalproex, dextromethorphan, gabapentin, lamotrigine, oxcarbazepine, pregabalin, tiagabine, topimarate, or valproate.
38. The method of embodiment 28, wherein the selective serotonin reuptake inhibitor is citalopram, escitalopram, fluoxetine, fluvoxamine, paroxetine, or sertraline.
39. The method of embodiment 28, wherein the serotonin-norepinephrine reuptake inhibitor is atomoxetine, duloxetine, desvenlafaxine, levomilnacipran, milnacipran, sibutramine, tramadol, or venlafaxine.
40. The method of embodiment 28, wherein the antidepressant is citalopram, fluoxetine, paroxetine, sertraline, escitalopram, trazodone, venlafaxine, bupropion, duloxetine, amitriptyline, venlafaxine, mirtazapine, desvenlafaxine, or nortriptyline.
41. The method of embodiment 28, wherein the anti-anxiety agent is lorazepam, oxazepam, or buspirone.
42. The method of embodiment 28, wherein the antipsychotic agent is quetiapine, trazodone, promazine, aripiprazole, ziprasidone, olanzapine, or risperidone.
43. The method of embodiment 28, wherein the antihistamine is acrivastine, azelastine, bilastine, bromodiphenhydramine, brompheniramine, buclizine, barbinoxamine, cetirizine, chlorodiphenhydramine, chlorpheniramine, clemastine, cyclizine, cyproheptadine, desloratadine, dexbrompheniramine, dexchlorpheniramine, dimenhydrinate dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, cimetidine, famotidine, lafutidine, nizatidine, ranitidine, roxatidine, tiotidine, clobenpropit, ABT-239, ciproxifan, conessine, A-349,821, thioperamide, thioperamide, JNJ 7777120, and VUF-6002.
44. The method of embodiment 28, wherein the benzodiazepine is quazepam, chlordiazepoxide, flurazepam, alprazolam, clorazepate, diazepam, estazolam, clonazepam, oxazepam, triazolam, lorazepam, temazepam, clobazam, and midazolam.
45. The method of embodiment 28, wherein the hormonal agent is oxytocin, estrogen, or testosterone.
46. The method of any one of embodiments 28-45, wherein the cyclobenzaprine or salt thereof and the one or more agents are in the same dosage form or in separate dosage forms packaged together or packaged separately, wherein the cyclobenzaprine or salt thereof and the one or more therapeutic agents are administered simultaneously or sequentially.
47. The method of embodiment 1, wherein the female subject has female genital organs by birth, reconstructive surgery or sex reassignment surgery.
48. The method of embodiment 47, wherein the female subject is premenopausal, perimenopausal, or postmenopausal.
49. The method of any one of embodiments 1-48, wherein the sexual dysfunction is further associated with the use of one or more agents selected from a group consisting of an antidepressant, an anxiolytic, an antihypertensive agent, a chemotherapy agent, a hormonal agent, a corticosteroid agent, an antipsychotic, an antihistamine, a benzodiazepine, a psychoactive agent, a barbiturate, lithium, an antihypertensive agent, an antilipid agent, a gonadotropin-releasing hormone (GnRh) agonist, a contraceptive, an anticholinergic agent, an amphetamine, an anorexic agent, and a narcotic agent.

### DETAILED DESCRIPTION

The present disclosure provides in some embodiments methods and pharmaceutical compositions for treating sexual dysfunction and associated symptoms in a subject in need or at risk thereof, wherein the pharmaceutical compositions comprise a therapeutically effective amount of cyclobenzaprine and a pharmaceutically acceptable carrier, optionally in combination with one or more therapeutic or non-therapeutic agents.

### Definitions

The term “herein” means the entire application.

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification can be combined with one or more other embodiments of this disclosure, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, the term “about” refers to a value or parameter that includes (and describes) embodiments that are directed to that value or parameter per se. For example, description referring to “about X” includes description of “X”. As used herein, the term “about” permits a variation of ±10% within the range of the significant digit. Numeric ranges are inclusive of the numbers defining the range.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Exemplary methods and materials are described herein, although methods and materials similar or equivalent to those described herein can also be used in the practice or testing of the various aspects and embodiments of this disclosure. The materials, methods, and examples are illustrative only and not intended to be limiting.

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “treat” and its cognates refer to a full or partial amelioration, improvement, or modulation of sexual dysfunction or at least one discernible symptom therein. In some embodiments, “treat at least one discernible symptom” refers to an improvement of desire and/or interest. In some embodiments, “treat” refers to an improvement of pleasure. In some embodiments, “treat of at least one discernible symptom” refers to improvement in desire and/or frequency. In some embodiments, “treat” refers to an improvement in arousal and/or excitement. In some embodiments, “treat at least one discernible symptom” refers to improvement in orgasm and/or completion. In some embodiments, “treat at least one discernible symptom” refers to improvement in a desire disorder. In some embodiments, “treat at least one discernible symptom” refers to improvement in an arousal disorder. In some embodiments, “treat at least one discernible symptom” refers to improvement in an orgasm disorder. In some embodiments, “treat at least one discernible symptom” refers to improvement in a sexual pain disorder.

In some embodiments of this disclosure, the cyclobenzaprine is an acid salt of cyclobenzaprine. In other embodiments, the acid salt is cyclobenzaprine HCl.

In other embodiments, the acid salt is combined with a basifying agent. In some embodiments, the basifying agent is an ingredient (and excipient) in a tablet or other formulation, and the basifying agent exerts its effects during the time the formulation is being dispersed in the mucous material, including buccal and sublingual tissue, while parts of the formulation are dissolving in the mucous material and for a period of time after the tablet is dissolved in the mucous material.

The “basifying agent” included in some embodiments of this disclosure is selected from a group consisting of potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, disodium citrate, trisodium citrate, borate, hydroxide, silicate, nitrate, dissolved ammonia, the conjugate bases of some organic acids (including bicarbonate), and sulfide. In some embodiments, the basifying agent is dipotassium hydrogen phosphate (K₂HPO₄), potassium dihydrogen phosphate (KH₂PO₄), disodium hydrogen phosphate (Na₂HPO₄), tripotassium citrate or trisodium citrate. A basifying agent that is particularly useful in combination with cyclobenzaprine HCl is dipotassium hydrogen phosphate (K₂HPO₄). Another basifying agent that is particularly useful in combination with cyclobenzaprine HCl is potassium dihydrogen phosphate (KH₂PO₄). Another basifying agent that is particularly useful in combination with cyclobenzaprine HCl is disodium hydrogen phosphate (Na₂HPO₄). Another basifying agent that is particularly useful in combination with cyclobenzaprine HCl is tripotassium citrate. Another basifying agent that is particularly useful in combination with cyclobenzaprine HCl is trisodium citrate.

In some embodiments of this disclosure, the cyclobenzaprine or its acid salt is present in a eutectic. In some embodiments, the eutectic includes mannitol. In some aspects, the mannitol is beta mannitol. In other embodiments, the mannitol is delta mannitol. In some aspects, the eutectic is a eutectic of the cyclobenzaprine HCl and mannitol is selected from the group consisting of a 75%±2% cyclobenzaprine HCl and 25%±2% mannitol eutectic, a 65%±2% cyclobenzaprine HCl and 35%±2% 8-mannitol eutectic, a mixture of a 75%±2% cyclobenzaprine HCl and 25%±2% β-mannitol and a 65%±2% cyclobenzaprine HCl and 35%±2% 8-mannitol eutectic, and a granule comprising an outer layer of a 65%±2% cyclobenzaprine HCl and 35%±2% δ-mannitol eutectic and an inner layer of β-mannitol. See, e.g., WO2014/145156 and WO2016/044796, both incorporated herein by reference. It should be understood that the “cyclobenzaprine HCl” eutectic of this disclosure refers to any of these eutectics or granules. In some aspects, the eutectic is combined with a basifying agent. See, e.g., WO2013/188847, incorporated herein by reference.

As used herein, the term a “eutectic” or “in the form of a eutectic” refers to a mixture of chemical compounds or elements that has a single chemical composition that melts at a lower temperature than any other composition made up of the same ingredients. A composition comprising a eutectic is known as the eutectic composition and its melting temperature is known as the eutectic temperature. Eutectic compositions often have a higher stability and/or dissolution rates than their non-eutectic counterparts. Because eutectics enhance dissolution, they can be employed to increase permeability in solid dispersions and dispersion systems.

### Method for Treating

In some embodiments, the present disclosure provides a method for treating, improving and/or preventing sexual dysfunction and associate symptoms thereof, comprising administering to a female subject in need or at risk thereof, a pharmaceutical composition comprising a therapeutically effective amount of cyclobenzaprine and a pharmaceutically acceptable carrier.

In some embodiments, the method for treating improving and/or preventing sexual dysfunction comprises administering a pharmaceutical composition comprising a pharmaceutically acceptable acid salt of cyclobenzaprine and a basifying agent. In some embodiments, the pharmaceutical composition comprises a eutectic of a pharmaceutically acceptable salt of cyclobenzaprine and mannitol, which optionally is combined with a basifying agent. The composition of this disclosure may be administered in one, two or more daily doses. In some embodiments, the method for treating and/or preventing sexual dysfunction comprises administering a daily dose between 0.1 mg and 30 mg of cyclobenzaprine or a pharmaceutically acceptable salt thereof. In some embodiments, the daily dose is between 1 mg and 20 mg of cyclobenzaprine or a pharmaceutically acceptable sale thereof. In some embodiments, the daily dose is less than 10 mg of cyclobenzaprine or a pharmaceutically acceptable salt thereof. In some embodiments, the daily dose is less than 5 mg of cyclobenzaprine or a pharmaceutically acceptable salt thereof. In some embodiments, the daily dose comprises about 5.6 mg of cyclobenzaprine or a pharmaceutically acceptable salt thereof. In some embodiments, the daily dose comprises about 5.6 mg of cyclobenzaprine HCl. In some embodiments, the daily dose comprises about 2.8 mg of cyclobenzaprine or a pharmaceutically acceptable salt thereof. In some embodiments, the daily dose comprises about 2.8 mg of cyclobenzaprine HCl. In some embodiments, the daily dose comprises 5.6 mg of cyclobenzaprine or a pharmaceutically acceptable salt thereof. In some embodiments, the daily dose comprises 5.6 mg of cyclobenzaprine HCl. In some embodiments, the daily dose comprises 2.8 mg of cyclobenzaprine or a pharmaceutically acceptable salt thereof. In some embodiments, the daily dose comprises 2.8 mg of cyclobenzaprine HCl. In some embodiments, the method for treating and/or preventing sexual dysfunction comprises administering simultaneously or sequentially two dosage units of cyclobenzaprine, and wherein each dosage unit comprises about 2.8 mg of cyclobenzaprine or a pharmaceutically acceptable salt thereof. In some embodiments, each of the two dosage units comprises about 2.8 mg of cyclobenzaprine HCl. In some embodiments, the method for treating and/or preventing sexual dysfunction comprises administering simultaneously or sequentially two dosage units of cyclobenzaprine, and wherein each dosage unit comprises 2.8 mg of cyclobenzaprine or a pharmaceutically acceptable salt thereof. In some embodiments, each of the two dosage units comprises 2.8 mg of cyclobenzaprine HCl. In some embodiments, the method for treating and/or preventing sexual dysfunction comprises administering a therapeutically effective amount of cyclobenzaprine or a pharmaceutically acceptable salt thereof daily. In some embodiments, the method for treating and/or preventing sexual dysfunction comprises administering a therapeutically effective amount of cyclobenzaprine or a pharmaceutically acceptable salt thereof once daily.

In some embodiments, the method for treating and/or preventing sexual dysfunction comprises administering a pharmaceutical composition comprising a therapeutically effective amount of cyclobenzaprine or a pharmaceutically acceptable salt thereof, wherein the pharmaceutical composition is formulated for sublingual, buccal, oral, suppository, intravenous, intramuscular, subcutaneous, inhalational, intranasal, thin film, transdermal, parenteral, rectal, or vaginal administration. In some embodiments, the method for treating and/or preventing sexual dysfunction comprises administering a pharmaceutical composition comprising a therapeutically effective amount of cyclobenzaprine or a pharmaceutically acceptable salt thereof, wherein the pharmaceutical composition is formulated for sublingual, buccal, oral, intravenous, intramuscular, subcutaneous, inhalational, intranasal, transdermal, parenteral, rectal, or vaginal administration. In some embodiments, the pharmaceutical composition is formulated for sublingual administration. In some embodiments, the pharmaceutical composition is formulated for buccal administration. In some embodiments, the pharmaceutical composition is formulated for oral administration. In some embodiments, the pharmaceutical composition is formulated for suppository administration. In some embodiments, the pharmaceutical composition is formulated for intravenous administration. In some embodiments the pharmaceutical composition is formulated for intramuscular administration. In some embodiments, the pharmaceutical composition is formulated for subcutaneous administration. In some embodiments, the pharmaceutical composition is formulated for intranasal administration. In some embodiments, the pharmaceutical composition is formulated for transdermal administration. In some embodiments, the pharmaceutical composition is formulated for parenteral administration. In some embodiments, the pharmaceutical composition is formulated for rectal administration. In some embodiments, the pharmaceutical composition is formulated for vaginal administration. In some embodiments, the pharmaceutical composition is formulated as a suppository or a thin film dosage form. In some embodiments, the pharmaceutical composition is formulated as a suppository dosage form. In some embodiments, the pharmaceutical composition is formulated as a thin film dosage form.

In some embodiments, the methods of this disclosure further comprises administering sequentially or simultaneously, with a composition of this disclosure comprising a cyclobenzaprine or pharmaceutically acceptable salt thereof, i.e., in combination with that composition, one or more therapeutic agents selected from the group consisting an estrogen receptor modulator, a 5-hydroxytryptamine 1A (5-HT_{1A}) receptor agonist, a 5-hydroxytryptamine 2A (5-HT_{2A}) antagonist, a synthetic or gonadal steroid agent, a phosphodiesterase inhibitor, a melanocortin receptor agonist, an alpha-1-adrenergic receptor antagonist, a beta-adrenergic antagonist, an anticonvulsant or a mood stabilizer, a selective serotonin reuptake inhibitor, a serotonin-norepinephrine reuptake inhibitor, an antidepressant, an anti-anxiety agent, an antipsychotic, an antihistamine, a benzodiazepine, a psychoactive agent, a barbiturate, lithium, an antihypertensive agent, an antilipid agent, a hormonal agent, a gonadotropin-releasing hormone (GnRh) agonist, a contraceptive agent, an anticholinergic agent, an amphetamine, a dopaminergic receptor agonist, an anorexic agent, and a narcotic agent. In some embodiments, the one or more therapeutic agents are administered sequentially or simultaneously with a composition of this disclosure comprising cyclobenzaprine HCl.

In some embodiments, the sexual dysfunction or risk thereof is in a female subject. In some embodiments, the subject has female genital organs by birth, reconstructive surgery or sex reassignment surgery. In some embodiments, the female subject is premenopausal, perimenopausal, or postmenopausal.

In some embodiments, the sexual dysfunction may be associated with the use of one or more agents selected from a group consisting of an antidepressant, an anxiolytic, an antihypertensive agent, a chemotherapy agent, a hormonal agent, a corticosteroid agent, an antipsychotic, an antihistamine, a benzodiazepine, a psychoactive agent, a barbiturate, lithium, an antihypertensive agent, an antilipid agent, a gonadotropin-releasing hormone (GnRh) agonist, a contraceptive, an anticholinergic agent, an amphetamine, an anorexic agent, and a narcotic agent.

In some embodiments, the sexual dysfunction is associated with one or more mental health conditions selected from the group consisting of a psychological condition, a mood disorder, a trauma and stressor related disorder, a personality disorder, a somatic symptom disorder, and an obsessive compulsive disorder. In some embodiments, the sexual dysfunction is associated with one or more mental health conditions selected from the group consisting of a psychological condition, a mood disorder, a trauma and stressor related disorder, a personality disorder, a somatic symptom disorder, and an obsessive compulsive disorder, wherein the psychological condition is neglect, wherein the mood disorder is a substance-related and addictive disorder, and wherein the trauma and stressor related disorder is a disinhibited social engagement disorder. In some embodiments, the psychological condition is neglect. In some embodiments, mood disorder is a substance-related and addictive disorder. In some embodiments, the trauma and stress related disorder is a disinhibited social engagement disorder.

### Changes in Sexual Functioning Questionnaire Short Form (CSFQ-14)

The Changes in Sexual Functioning Questionnaire (CSFQ) is a 36-item clinical and research instrument identifying five scales of sexual functioning. The CSFQ has been shortened to a factor structure of a 14-item version (CSFQ-14), which yields scores for three scales corresponding to the phases of the sexual response cycle (e.g., desire, arousal, and orgasm) as well as the five scales of the original CSFQ (e.g., desire/frequency, desire/interest, arousal/excitement, orgasm/complete, and pleasure). Factor analysis confirms the construct validity of the CSFQ-14 as a global measure of sexual dysfunction.

### DSM-5 Diagnostic Criteria for PTSD and CAPS-5

The Diagnostic and Statistical Manual of Mental Disorders, Fifth Edition (DSM-5) is a diagnostic tool published by the American Psychiatric Association. DSM-5 contains descriptions, symptoms and criteria for diagnosing mental disorders. It also contains common language for clinicians to communicate about their patients to establish consistent and reliable diagnoses that can be used in the research of mental disorders. DSM-5 further provides researchers common language to study the criteria for potential future revisions and to aid the development of medications and other interventions.

The Clinician-Administered PTSD Scale (CAPS) is a semi-structured diagnostic interview that assesses essential features of PTSD as defined by the DSM-5 Diagnostic Criteria for PTSD (Weathers et al., 2017). It can also be used to assess associated features of the diagnostic syndrome (e.g., survivor guilt). The interview is designed to accommodate different time spans post-trauma as the reference point for diagnosis. The CAPS affords the clinician flexibility to inquire about symptoms and diagnostic status over the past week, most recent month, and/or for lifetime diagnosis. Any one, or all three, of the time frames may be used depending on the nature task at hand. Other diagnostic scales or tool based on the DSM-5 Diagnostic Criteria for diagnosing PTSD are also well-known. They include, for example, a PTSD checklist for DSM-5 (PCL-5), a clinician-completed symptom severity, intensity and/or frequency rating scale, and a patient-completed symptom severity, intensity and/or intensity rating scale.

In some embodiments, assessing changes in one or more of the DSM-5 Diagnostic Criteria items for PTSD is based on one or more of a Clinician-Administered PTSD Scale for DSM-5 (CAPS-5), a PTSD checklist for DSM-5 (PCL-5), a clinician-completed symptom severity, intensity, and/or frequency rating scale, or a patient-completed symptom severity, intensity, and/or frequency rating scale. The Clinician-Administered PTSD Scale for DSM-5 (CAPS-5) is a 30-item questionnaire corresponding to the DSM-5 diagnosis for PTSD. CAPS-5 requires the identification of a single index trauma to serve as the basis of symptom inquiry. CAPS-5 asks questions relevant to assessing the dissociative subtype of PTSD (depersonalization and derealization), but no longer includes other associated symptoms (e.g., gaps in awareness). CAPS-5 symptom severity ratings are based on symptom frequency and intensity. However, CAPS-5 items are rated with a single severity score in contrast to previous versions of the CAPS which required separate frequency and intensity scores.

### Pharmaceutical Compositions and Combinations

In some embodiments, the present disclosure provides a pharmaceutical composition comprising a therapeutically effective amount of cyclobenzaprine or a pharmaceutically acceptable salt thereof of this disclosure and optionally as a combination therewith one or more therapeutic agents selected from the group consisting of an estrogen receptor modulator, a 5-hydroxytryptamine 1A (5-HT_{1A}) receptor agonist, a steroid agent, a phosphodiesterase inhibitor, a melanocortin receptor agonist, an alpha-1-adrenergic receptor antagonist, a beta-adrenergic antagonist, an anticonvulsant or a mood stabilizer, a selective serotonin reuptake inhibitor, a serotonin-norepinephrine reuptake inhibitor, an antidepressant, an anti-anxiety agent, an antipsychotic, an antihistamine, a benzodiazepine, a psychoactive agent, a barbiturate, lithium, an antihypertensive agent, an antilipid agent, a hormonal agent, a gonadotropin-releasing hormone (GnRh) agonist, a contraceptive, an anticholinergic, an amphetamine, an anorexic agent, and a narcotic agent. In some embodiments, the estrogen receptor modulator is ospemifene. In some embodiments, the 5-HT_{1A} receptor agonist or the 5-HT_{2A} receptor agonist is flibanserin. In some embodiments, the dopaminergic receptor agonist is apomorphine. In some embodiments, the steroid agent is tibolone, estrogen, or testosterone. In some embodiments, the phosphodiesterase inhibitor is sildenafil or tadalafil. In some embodiments, the melanocortin receptor agonist is bremelanotide. In some embodiments, the alpha-1-adrenergic receptor antagonist is prazosin, terazosin, doxazosin, silodosin, alfuzosin, or tamsulosin. In some embodiments, the beta adrenergic receptor antagonist is propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, ICI-118,551, SR 59230A, or nebivolol. In some embodiments, the anticonvulsant or mood stabilizer is carbamazepine, divalproex, dextromethorphan, gabapentin, lamotrigine, oxcarbazepine, pregabalin, tiagabine, topimarate, or valproate. In some embodiments, the selective serotonin reuptake inhibitor is citalopram, escitalopram, fluoxetine, fluvoxamine, paroxetine, or sertraline. In some embodiments, the serotonin-norepinephrine reuptake inhibitor is atomoxetine, duloxetine, desvenlafaxine, levomilnacipran, milnacipran, sibutramine, tramadol, or venlafaxine. In some embodiments, the antidepressant is citalopram, fluoxetine, paroxetine, sertraline, escitalopram, trazodone, venlafaxine, bupropion, duloxetine, amitriptyline, venlafaxine, mirtazapine, desvenlafaxine, or nortriptyline. In some embodiments, the anti-anxiety agent is lorazepam, oxazepam, or buspirone. In some embodiments, the antipsychotic agent is quetiapine, trazodone, promazine, aripiprazole, ziprasidone, olanzapine, or risperidone. In some embodiments, the antihistamine is acrivastine, azelastine, bilastine, bromodiphenhydramine, brompheniramine, buclizine, barbinoxamine, cetirizine, chlorodiphenhydramine, chlorpheniramine, clemastine, cyclizine, cyproheptadine, desloratadine, dexbrompheniramine, dexchlorpheniramine, dimenhydrinate dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, cimetidine, famotidine, lafutidine, nizatidine, ranitidine, roxatidine, tiotidine, clobenpropit, ABT-239, ciproxifan, conessine, A-349,821, thioperamide, thioperamide, JNJ 7777120, and VUF-6002. In some embodiments, the benzodiazepine is quazepam, chlordiazepoxide, flurazepam, alprazolam, clorazepate, diazepam, estazolam, clonazepam, oxazepam, triazolam, lorazepam, temazepam, clobazam, and midazolam. In some embodiments, the hormonal agent is oxytocin, estrogen, or testosterone.

In some embodiments, the cyclobenzaprine or a pharmaceutically acceptable salt thereof of this disclosure and the one or more optional therapeutic agents of the combination are in the same dosage form or in separate dosage forms packaged together or packaged separately, wherein the cyclobenzaprine or a pharmaceutically acceptable salt thereof and the optional one or more therapeutic agents are administered simultaneously or sequentially. In some embodiments, the cyclobenzaprine or salt and the one or more optional therapeutic agents are in the same dosage form. In some embodiments, the cyclobenzaprine and the one or more optional therapeutic agents are in the separate dosage form. In some embodiments, the cyclobenzaprine or salt and the one or more optional therapeutic agents are packaged together. In some embodiments, the cyclobenzaprine or salt and the one or more optional therapeutic agents are packaged separately. In some embodiments, the cyclobenzaprine or salt and the one or more optional therapeutic agents are administered simultaneously. In some embodiments, the cyclobenzaprine or salt and the one or more optional therapeutic agents are administered sequentially.

In some embodiments, the one or more optional therapeutic agents is ospemifene, flibanserin, tibolone, estrogen, or testosterone, sildenafil, bremelanotide, prazosin, terazosin, doxazosin, silodosin, alfuzosin, tamsulosin, propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, ICI-118,551, SR 59230A, nebivolol, carbamazepine, divalproex, dextromethorphan, gabapentin, lamotrigine, oxcarbazepine, pregabalin, tiagabine, topimarate, valproate, citalopram, escitalopram, fluoxetine, fluvoxamine, paroxetine, sertraline, atomoxetine, duloxetine, desvenlafaxine, levomilnacipran, milnacipran, sibutramine, tramadol, venlafaxine, citalopram, fluoxetine, paroxetine, sertraline, escitalopram, trazodone, venlafaxine, bupropion, duloxetine, amitriptyline, venlafaxine, mirtazapine, desvenlafaxine, nortriptyline, lorazepam, oxazepam, buspirone, quetiapine, trazodone, promazine, aripiprazole, ziprasidone, olanzapine, risperidone, acrivastine, azelastine, bilastine, bromodiphenhydramine, brompheniramine, buclizine, barbinoxamine, cetirizine, chlorodiphenhydramine, chlorpheniramine, clemastine, cyclizine, cyproheptadine, desloratadine, dexbrompheniramine, dexchlorpheniramine, dimenhydrinate dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, cimetidine, famotidine, lafutidine, nizatidine, ranitidine, roxatidine, tiotidine, clobenpropit, ABT-239, ciproxifan, conessine, A-349,821, thioperamide, thioperamide, JNJ 7777120, VUF-6002, quazepam, chlordiazepoxide, flurazepam, alprazolam, clorazepate, diazepam, estazolam, clonazepam, oxazepam, triazolam, lorazepam, temazepam, clobazam, midazolam, or oxytocin.

In some embodiments, the cyclobenzaprine is the free base or a pharmaceutically acceptable salt of the free base. In some embodiments, the cyclobenzaprine is the free base. In some embodiments, the cyclobenzaprine is a pharmaceutically acceptable salt. In some embodiments, the cyclobenzaprine is an acid salt. In some embodiments, the cyclobenzaprine acid salt is cyclobenzaprine hydrochloride. In some embodiments, the pharmaceutical composition comprises a pharmaceutically acceptable salt of cyclobenzaprine and a basifying agent.

In some embodiments, the composition of this disclosure comprises between 0.1 mg and 30 mg of cyclobenzaprine or a pharmaceutically acceptable salt thereof. In some embodiments, the composition comprises between 1 mg and 20 mg of cyclobenzaprine or a pharmaceutically acceptable salt thereof. In some embodiments, the composition comprises less than 10 mg of cyclobenzaprine or a pharmaceutically acceptable salt thereof. In some embodiments, the composition comprises less than 5 mg of cyclobenzaprine or a pharmaceutically acceptable salt thereof. In some embodiments, the composition comprises about 5.6 mg of cyclobenzaprine or a pharmaceutically acceptable salt thereof. In some embodiments, the composition comprises about 2.8 mg of cyclobenzaprine or a pharmaceutically acceptable salt thereof. In some embodiments, the composition comprises about 5.6 mg of cyclobenzaprine HCl. In some embodiments, the composition comprises about 2.8 mg of cyclobenzaprine HCl. In some embodiments, the composition comprises 5.6 mg of cyclobenzaprine or a pharmaceutically acceptable salt thereof. In some embodiments, the composition comprises 2.8 mg of cyclobenzaprine or a pharmaceutically acceptable salt thereof. In some embodiments, the composition comprises 5.6 mg of cyclobenzaprine HCl. In some embodiments, the composition comprises 2.8 mg of cyclobenzaprine HCl.

In some embodiments, the pharmaceutical composition of this disclosure is formulated for sublingual, buccal, oral, suppository, intravenous, intramuscular, subcutaneous, inhalational, intranasal, thin film, transdermal, parenteral, rectal, or vaginal administration. In some embodiments, the pharmaceutical composition of this disclosure is formulated for sublingual, buccal, oral, intravenous, intramuscular, subcutaneous, inhalational, intranasal, transdermal, parenteral, rectal, or vaginal administration. In some embodiments, the pharmaceutical composition is formulated for sublingual administration. In some embodiments, the pharmaceutical composition is formulated for buccal administration. In some embodiments, the pharmaceutical composition is formulated for oral administration. In some embodiments, the pharmaceutical composition is formulated for suppository administration. In some embodiments, the pharmaceutical composition is formulated for intravenous administration. In some embodiments, the pharmaceutical composition is formulated for intramuscular administration. In some embodiments, the pharmaceutical composition is formulated for subcutaneous administration. In some embodiments, the pharmaceutical composition is formulated for inhalational administration. In some embodiments, the pharmaceutical composition is formulated for intranasal administration. In some embodiments, the pharmaceutical composition is formulated for transdermal administration. In some embodiments, the pharmaceutical composition is formulated for parenteral administration. In some embodiments, the pharmaceutical composition is formulated for rectal administration. In some embodiments, the pharmaceutical composition is formulated for vaginal administration. In some embodiments, the pharmaceutical composition of this disclosure is formulated as a suppository or a thin film. In some embodiments, the pharmaceutical composition of this disclosure is formulated as a suppository. In some embodiments, the pharmaceutical composition of this disclosure is formulated as a thin film.

In some embodiments, the pharmaceutical composition of this disclosure is administered sublingually, buccally, orally, intravenously, intramuscularly, subcutaneously, inhalationally, intranasally, transdermally, parenterally, rectally, or vaginally. In some embodiments, the pharmaceutical composition of this disclosure is administered sublingually. In some embodiments, the pharmaceutical composition of this disclosure is administered buccally. In some embodiments, the pharmaceutical composition of this disclosure is administered orally. In some embodiments, the pharmaceutical composition of this disclosure is administered intravenously. In some embodiments, the pharmaceutical composition of this disclosure is administered intramuscularly. In some embodiments, the pharmaceutical composition of this disclosure is administered subcutaneously. In some embodiments, the pharmaceutical composition of this disclosure is administered inhalationally. In some embodiments, the pharmaceutical composition of this disclosure is administered intranasally. In some embodiments, the pharmaceutical composition of this disclosure is administered transdermally. In some embodiments, the pharmaceutical composition of this disclosure is administered parenterally. In some embodiments, the pharmaceutical composition of this disclosure is administered rectally. In some embodiments, the pharmaceutical composition of this disclosure is administered vaginally. In some embodiments, the pharmaceutical composition of this disclosure is administered in a suppository or a thin film dosage form. In some embodiments, the pharmaceutical composition of this disclosure is administered in a suppository. In some embodiments, the pharmaceutical composition of this disclosure is administered in a thin film dosage form.

In some embodiments, the dosage form of the compositions of this disclosure is a tablet, a film, a thin film, a liquid, powder, or a spray solution. In some embodiments, the dosage form is a tablet. In some embodiments, the dosage form is a film. In some embodiments, the dosage form is a thin film. In some embodiments, the dosage form is a liquid. In some embodiments, the dosage form is a powder. In some embodiments, the dosage form is a spray solution. In some embodiments, the dosage form is a sublingual tablet, a sublingual film, a sublingual liquid, sublingual powder, or a sublingual spray solution. In some embodiments, the dosage form is a sublingual tablet. In some embodiments, the dosage form is a sublingual film. In some embodiments, the dosage form is a sublingual liquid. In some embodiments, the dosage form is sublingual powder. In some embodiments, the dosage form is a sublingual spray solution.

### EXAMPLES

### Example 1

Effects on Female Sexual Functioning of a Low Dose, Sublingual Formulation of Cyclobenzaprine in Subjects with Military-Related PTSD after 12 Weeks of Treatment

Methods: In this 12-week, multicenter, adaptive design, randomized controlled fixed-dose trial, 5.6 mg cyclobenzaprine HCl (2×2.8 mg sublingual tablets, comprising a 75%±2% cyclobenzaprine HCl and 25%±2% mannitol eutectic and a potassium phosphate dibasic anhydrous basifying agent) taken daily at bedtime was compared to placebo for the treatment of PTSD at 45 U.S. sites. Eligible participants (males and females) were 18-75 years of age, had experienced DSM-5 PTSD Criterion A-qualifying trauma(s) during military service since 2001, met DSM-5-defined PTSD by the Clinician-Administered PTSD Scale (CAPS-5), had a baseline CAPS-5 severity score ≥33, and were free of antidepressants, and free or washed off other psychotropic medications. A pre-planned interim analysis was conducted when about half the originally planned sample of 550 participants had outcome data.

The study was stopped early after the interim analysis indicated there was a low probability of achieving a significant separation between treatment groups on the CAPS-5 primary endpoint. Thus, all analyses were conducted on the interim analysis sample of 274 participants. The modified intention-to-treat (mITT) population, which included all randomized subjects with at least one post-baseline CAPS-5, included 125 participants treated with 5.6 mg dose per day the above cyclobenzaprine HCl sublingual formulation and 127 participants on placebo were studied. The female subgroup of the study, which included 10 participants treated with the 5.6 mg dose per day the above cyclobenzaprine HCl sublingual formulation and 17 participants on placebo, demonstrated a large observed mean improvement in CAPS-5 over placebo (by −9.1 units after 12 weeks of treatment).

Sexual functioning was measured by Changes in Sexual Functioning Questionnaire short-form (CSFQ-14) which is a validated 14-item measure (Keller, McGarvey, Clayton, 2006). Parallel gender-specific versions are administered to males (CSFQ-14-M) and females (CSFQ-14-F), and each sex was analyzed separately. In addition to a total sexual functioning score, there are five sub-scales: desire/frequency, desire/interest, arousal/excitement, orgasm/complete, and pleasure. The scales range from (never) to (every day) and higher scores reflect greater levels of sexual functioning.

Moderate effect sizes were found on the CSFQ-14-F total score and on 4 of 5 subscales between females treated with the 5.6 mg dose (N=8) and placebo (N=16). Due to sample size differences between the active treatment and placebo females groups, the results are better characterized by Hedge's g effect sizes. Specifically, there was a moderate effect on the total score g=0.49 (95% CI-1.34, 0.37), desire/interest subscale g=0.63 (95% CI-1.49, 0.25), pleasure subscale g=0.54 (95% CI-1.40, 0.33), desire/frequency subscale g=0.46 (95% CI-1.31, 0.40) and arousal/excitement subscale g=0.55 (95% CI-1.41, 0.32). No effect was found on the orgasm/completion scale. Overall, adverse events (AE) in the study were comparable to prior studies with a similar 5.6 mg cyclobenzaprine HCl dose. The most frequent AE was oral hypoaesthesia (tongue/mouth numbness), related to the site of administration, which was generally transient (<60 min post administration) and never rated as severe. The most common systemic AE was somnolence, also never rated as severe. Two participants on placebo and 8 participants who received the 5.6 mg cyclobenzaprine HCl dose, had at least one AE leading to study discontinuation.

The results in the female group suggests that the 5.6 mg daily dose of the cyclobenzaprine HCl composition has a clinically meaningful effect on improvement of overall sexual functioning, the frequency of sexual acts, interest in sexual experiences, ease of arousal, and current enjoyment of sex over placebo in females with military-related PTSD. This effect was strong for arousal/excitement, which is a primary form of female sexual dysfunction with limited effective and safe treatment options available (Goldstein, 2000).

### Example 2

Effects on Female Sexual Functioning of a Sublingual Formulation of Cyclobenzaprine HCl in Military and Civilian Phase 3 PTSD Trials: Preliminary Evidence for a Female-Specific Improvement in Sexual Functioning after 12 Weeks

Three randomized, placebo-controlled and double-blind clinical trials of the above described 5.6 mg cyclobenzaprine HCl sublingual formulation (2×2.8 mg tablets) were performed, a Phase 2 study and a Phase 3 study in military-related PTSD, and a Phase 3 study in predominantly civilian PTSD. All three trials showed encouraging activity using the 5.6 mg cyclobenzaprine HCl dose on clinician- and patient-rated global PTSD symptoms (Clinician Global Impression [CGI] and Patient Global Impression of Change [PGIC]). Given the very low rates of adverse events related to sexual function in both drug and placebo groups in the Phase 2 study, a systematic study was undertaken to assess the effects of the treatment on female sexual functioning in subsequent Phase 3 studies. The present retrospective analysis examined the activity of the cyclobenzaprine HCl sublingual formulation on the Changes in Sexual Functioning Questionnaire short form (CSFQ-14) in the two Phase 3 studies.

In the Phase 3 study in military-related PTSD, eligible participants (males and females) were 18-75 years of age, had experienced DSM-5 PTSD Criterion A-qualifying trauma(s) during military service since 2001, met DSM-5-defined PTSD by the CAPS-5, had a baseline CAPS-5 severity score ≥33, and were free of antidepressants, and free or washed off other psychotropic medications. In the Phase 3 study in predominantly civilian PTSD, the inclusion criteria were broadened to include civilian participants with current PTSD, as determined by the CAPS-5. Accordingly, this study included 94% civilian trauma with a minimum baseline severity score of ≥33 on the CAPS-5. Both studies were stopped early after the interim analysis indicated there was a low probability of achieving a significant separation between treatment groups on the CAPS-5 primary endpoint. Thus, analyses were conducted on the interim analysis sample of 252 participants in the military Phase 3 study (89% male [n=225 M; n=27 F]; 100% military PTSD) and 143 subjects in the civilian Phase 3 study (79% female [n=129 F; n=34 M]; 94% civilian PTSD).

The CSFQ-14 is a validated 14-item measure (Keller, McGarvey, Clayton, 2006) that has male and female versions which were analyzed separately. In addition to a total sexual functioning score, there are five sub-scales: desire/frequency, desire/interest, arousal/excitement, orgasm/complete, and pleasure. The items range on 5-point Likert scale from (never) to (every day). Higher scores reflect greater levels of sexual functioning.

In the Phase 3 study of predominantly male and all military-related PTSD, the sample size (N=24) of female completers was small and the study was not powered to detect differences in subgroups. Therefore, effect sizes, as characterized by Hedges' g, are reported for females treated with the 5.6 mg cyclobenzaprine HCl dose (N=8) relative to placebo (N=16) on the CSFQ-14 total score and on 4 of 5 subscales: total score g=0.49 (95% CI-1.34, 0.37), desire/interest subscale g=0.63 (95% CI-1.49, 0.25), pleasure subscale g=0.54 (95% CI-1.40, 0.33), desire/frequency subscale g=0.46 (95% CI-1.31, 0.40) and arousal/excitement subscale g=0.55 (95% CI-1.41, 0.32). No effect was found on the orgasm/completion scale.

In the Phase 3 study of predominantly civilian PTSD subjects, the female completers in the 5.6 mg cyclobenzaprine HCl dose (N=58) group trended towards improvement on the CSFQ-14-F total score versus placebo (N=55), with a moderate effect size (p=0.07, Hedges' g=0.37 [95% CI 0.00, 0.74]). Although the study was not powered to detect differences in subgroups, small to moderate effect sizes were observed for each of the subscales including: desire/interest g=0.23 (95% CI-0.14, 0.60), desire/frequency g=0.21 (95% CI-0.16, 0.57), pleasure g=0.19 (95% CI-0.17, 0.56), arousal/excitement g=0.33 (95% CI-0.04, 0.70), and orgasm/completion g=0.29 (95% CI-0.08, 0.66).

Adverse events (AE) in both Phase 3 studies were comparable to prior studies with the 5.6 mg cyclobenzaprine HCl dose. The most frequent AE was oral hypoaesthesia (tongue/mouth numbness), related to the site of administration, which was generally transient (<60 minutes post administration) and never rated as severe. The most common systemic AE was somnolence, also never rated as severe.

Results in the female groups of the two Phase 3 studies suggest a clinically meaningful trend of improvement in overall sexual functioning in females with PTSD. Results from both studies demonstrated a strong effect for female arousal/excitement, which is a primary form of female sexual dysfunction with limited available treatment options. Although the effect size was larger in the military Phase 3 study, there were relatively few females in that study. In the civilian Phase 3 study, the female sample was larger, and proportionately more female participants in that study reported an index trauma related to sexual trauma.

These above examples indicate that the 5.6 mg cyclobenzaprine HCl dose of a composition of this disclosure has activity in female SD in military-related PTSD and civilian PTSD.

What is claimed is:

1. A method for treating or preventing sexual dysfunction and associated symptoms thereof, comprising administering to a female subject in need or at risk thereof, a pharmaceutical composition comprising a therapeutically effective amount of cyclobenzaprine and a pharmaceutically acceptable carrier, wherein the sexual dysfunction is associated with one or more mental health conditions selected from the group consisting of a psychological condition, wherein the psychological condition is neglect, a mood disorder, wherein the mood disorder is a substance related and addictive disorder, a trauma and stressor related disorder, wherein the trauma and stressor related disorder is a disinhibited social engagement disorder, a personality disorder, a somatic symptom disorder, and an obsessive compulsive disorder.

2. The method of embodiment 1, wherein the cyclobenzaprine is a free base or a pharmaceutically acceptable salt thereof.

3. The method of embodiment 1 or 2, wherein the pharmaceutically acceptable salt of cyclobenzaprine is a cyclobenzaprine acid salt.

4. The method of embodiment 3, wherein the cyclobenzaprine acid salt is cyclobenzaprine HCl.

5. The method of any one of embodiments 1-4, wherein the cyclobenzaprine or pharmaceutically salt thereof is in the form of a eutectic.

6. The method of embodiment 5, wherein the eutectic is a mannitol eutectic.

7. The method of embodiment 6, wherein the mannitol eutectic is selected for the group consisting of a 75%±2% cyclobenzaprine HCl and 25%±2% mannitol eutectic, a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol.

8. The method of any one of embodiments 1-7, wherein the composition comprising a pharmaceutically acceptable salt of cyclobenzaprine further comprises a basifying agent.

9. The method of embodiment 8, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

10. The method according to embodiment 9, wherein the basifying agent is dipotassium hydrogen phosphate.

11. The method of embodiment 1, wherein the composition comprises between 0.1 mg and 30 mg of cyclobenzaprine or a pharmaceutically acceptable salt thereof.

12. The method of embodiment 11, wherein the composition comprises between 1 mg and 20 mg of cyclobenzaprine or a pharmaceutically acceptable salt thereof.

13. The method of embodiment 12, wherein the composition comprises less than 10 mg of cyclobenzaprine or a pharmaceutically acceptable salt thereof.

14. The method of embodiment 12, wherein the composition comprises less than 5 mg of cyclobenzaprine or a pharmaceutically acceptable salt thereof.

15. The method of embodiment 13, wherein the composition comprises about 5.6 mg of cyclobenzaprine or a pharmaceutically acceptable salt thereof.

16. The method of embodiment 15, wherein the composition comprises about 5.6 mg of cyclobenzaprine HCl.

17. The method of embodiment 13, wherein the composition comprises about 2.8 mg of cyclobenzaprine or a pharmaceutically acceptable salt thereof.

18. The method of embodiment 17, wherein the composition comprises about 2.8 mg of cyclobenzaprine HCl.

19. The method of embodiment 17 or 18, wherein the composition is administered simultaneously or sequentially in two dosage units, and wherein the combined amount of the composition in the two dosage units is about 5.6 mg of cyclobenzaprine or a pharmaceutically acceptable salt.

20. The method of embodiment 19, wherein the composition is administered simultaneously in two dosage units, and wherein each dosage unit comprises about 2.8 mg of cyclobenzaprine or a pharmaceutically acceptable salt thereof.

21. The method of embodiment 19, wherein the composition is administered sequentially in two dosage units, and wherein each dosage unit comprises about 2.8 mg of cyclobenzaprine or a pharmaceutically acceptable salt thereof.

22. The method of any one of embodiments 1-21, wherein the pharmaceutical composition is administered daily.

23. The method of any one of embodiments 1-22, wherein the composition is administered once daily.

24. The method of embodiment 23, wherein the pharmaceutical composition is formulated for sublingual, buccal, oral, intravenous, intramuscular, subcutaneous, inhalational, intranasal, transdermal, parenteral, rectal, or vaginal administration.

25. The method of embodiment 23, wherein the pharmaceutical composition is formulated as a suppository or a thin film dosage form.

26. The method of embodiment 25, wherein the pharmaceutical composition is formulated as a thin film dosage form.

27. The method of embodiment 24 or 26, wherein the pharmaceutical composition is formulated for sublingual administration.

28. The method of any one of embodiments 1-27, wherein the method further comprises administering sequentially or simultaneously one or more therapeutic agents selected from the group consisting an estrogen receptor modulator, a 5-hydroxytryptamine 1A (5-HT_{1A}) receptor agonist, a 5-hydroxytryptamine 2A (5-HT_{2A}) antagonist, a synthetic or gonadal steroid agent, a phosphodiesterase inhibitor, a melanocortin receptor agonist, an alpha-1-adrenergic receptor antagonist, a beta-adrenergic antagonist, an anticonvulsant or a mood stabilizer, a selective serotonin reuptake inhibitor, a serotonin-norepinephrine reuptake inhibitor, an antidepressant, an anti-anxiety agent, an antipsychotic, an antihistamine, a benzodiazepine, a psychoactive agent, a barbiturate, lithium, an antihypertensive agent, an antilipid agent, a hormonal agent, a gonadotropin-releasing hormone (GnRh) agonist, a contraceptive agent, an anticholinergic agent, an amphetamine, a dopaminergic receptor agonist, an anorexic agent, and a narcotic agent.

29. The method of embodiment 28, wherein the estrogen receptor modulator is ospemifene.

30. The method of embodiment 28, wherein the 5-HT_{1A} receptor agonist or the 5-HT_{2A} receptor agonist is flibanserin.

31. The method of embodiment 28, wherein the dopaminergic receptor agonist is apomorphine.

32. The method of embodiment 28, wherein the steroid agent is tibolone, estrogen, or testosterone.

33. The method of embodiment 28, wherein the phosphodiesterase inhibitor is sildenafil or tadalafil.

34. The method of embodiment 28, wherein the melanocortin receptor agonist is bremelanotide.

35. The method of embodiment 28, wherein the alpha-1-adrenergic receptor antagonist is prazosin, terazosin, doxazosin, silodosin, alfuzosin, or tamsulosin.

36. The method of embodiment 28, wherein the beta adrenergic receptor antagonist is propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, ICI-118,551, SR 59230A, or nebivolol.

37. The method of embodiment 28, wherein the anticonvulsant or mood stabilizer is carbamazepine, divalproex, dextromethorphan, gabapentin, lamotrigine, oxcarbazepine, pregabalin, tiagabine, topimarate, or valproate.

38. The method of embodiment 28, wherein the selective serotonin reuptake inhibitor is citalopram, escitalopram, fluoxetine, fluvoxamine, paroxetine, or sertraline.

39. The method of embodiment 28, wherein the serotonin-norepinephrine reuptake inhibitor is atomoxetine, duloxetine, desvenlafaxine, levomilnacipran, milnacipran, sibutramine, tramadol, or venlafaxine.

40. The method of embodiment 28, wherein the antidepressant is citalopram, fluoxetine, paroxetine, sertraline, escitalopram, trazodone, venlafaxine, bupropion, duloxetine, amitriptyline, venlafaxine, mirtazapine, desvenlafaxine, or nortriptyline.

41. The method of embodiment 28, wherein the anti-anxiety agent is lorazepam, oxazepam, or buspirone.

42. The method of embodiment 28, wherein the antipsychotic agent is quetiapine, trazodone, promazine, aripiprazole, ziprasidone, olanzapine, or risperidone.

43. The method of embodiment 28, wherein the antihistamine is acrivastine, azelastine, bilastine, bromodiphenhydramine, brompheniramine, buclizine, barbinoxamine, cetirizine, chlorodiphenhydramine, chlorpheniramine, clemastine, cyclizine, cyproheptadine, desloratadine, dexbrompheniramine, dexchlorpheniramine, dimenhydrinate dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, quetiapine, rupatadine, tripelennamine, triprolidine, levocetirizine, desloratadine, pyrilamine, cimetidine, famotidine, lafutidine, nizatidine, ranitidine, roxatidine, tiotidine, clobenpropit, ABT-239, ciproxifan, conessine, A-349,821, thioperamide, thioperamide, JNJ 7777120, and VUF-6002.

44. The method of embodiment 28, wherein the benzodiazepine is quazepam, chlordiazepoxide, flurazepam, alprazolam, clorazepate, diazepam, estazolam, clonazepam, oxazepam, triazolam, lorazepam, temazepam, clobazam, and midazolam.

45. The method of embodiment 28, wherein the hormonal agent is oxytocin, estrogen, or testosterone.

46. The method of any one of embodiments 28-45, wherein the cyclobenzaprine or salt thereof and the one or more agents are in the same dosage form or in separate dosage forms packaged together or packaged separately, wherein the cyclobenzaprine or salt thereof and the one or more therapeutic agents are administered simultaneously or sequentially.

47. The method of embodiment 1, wherein the female subject has female genital organs by birth, reconstructive surgery or sex reassignment surgery.

48. The method of embodiment 47, wherein the female subject is premenopausal, perimenopausal, or postmenopausal.

49. The method of any one of embodiments 1-48, wherein the sexual dysfunction is further associated with the use of one or more agents selected from a group consisting of an antidepressant, an anxiolytic, an antihypertensive agent, a chemotherapy agent, a hormonal agent, a corticosteroid agent, an antipsychotic, an antihistamine, a benzodiazepine, a psychoactive agent, a barbiturate, lithium, an antihypertensive agent, an antilipid agent, a gonadotropin-releasing hormone (GnRh) agonist, a contraceptive, an anticholinergic agent, an amphetamine, an anorexic agent, and a narcotic agent.
